(19) United States
(12) Patent Application Publication
(10) Pub. No.: US 2008/0286253 A1
(43) Pub. Date: Nov. 20, 2008

(54) Composition Comprising Polymeric Material And Uses Thereof

(75) Inventors: Jérôme Mulhbacher, Sherbrooke (CA); Mircea Alexandru Mateescu, Montreal (CA); Carmen Calinescu, Pierrefonds (CA)

Correspondence Address: GOUDREAU GAGE DUBUC, 2000 MCGILL COLLEGE, SUITE 2200, MONTREAL, QC, H3A 3H3, CA

(73) Assignee: Transfert Plus Societe En Commandite, Montreal, QC (CA)

(21) Appl. No.: 10/597,462

(22) PCT Filed: Feb. 9, 2005
(86) PCT No.: PCT/CA2005/000160

§ 371 Date: Jul. 17, 2008

Related U.S. Application Data
(60) Provisional application No. 60/542,299, filed on Feb. 9, 2004.

(51) Int. Cl. A61K 47/26; A61K 38/02; A61K 35/74; A61K 38/47; A61K 38/48; A61K 38/43; C12N 1/04; A61P 1/00

(52) U.S. Cl. 424/93.45; 514/778; 514/2; 424/94.1; 424/93.1; 424/94.61; 424/94.64; 424/93.4; 424/93.48; 435/260

(57) ABSTRACT

Compositions for the selective delivery of an agent comprising an uncrosslinked starch modified by an acidic group and an agent are described. The compositions are substantially resistant to degradation and thus result in no or substantially no release of the agent in a first environment, and are capable of degradation in a second environment thereby allowing release of the agent. The pKa of the acidic group of the uncrosslinked starch used is higher than the pH of the first environment and lower than or equal to the pH of the subsequent environment. Also described herein are methods of preparing the compositions, methods of using the compositions and the uncrosslinked starch modified by an acidic group, and corresponding commercial packages.

### FIELD OF THE INVENTION

The invention relates to a composition and more particularly to a composition comprising a polymeric material.

### BACKGROUND OF THE INVENTION

Numerous polymeric matrices based on vinylic or acrylic polymers, polysaccharide, polylactic-glycolic acid (PGA) and several others polymers are largely used as excipients for oral drug formulations, ensuring drug transportation through gastrointestinal tractus (GIT) and delaying the release of the active agent over an extended period of time following oral intake. For instance, the recently introduced cross-linked high amylose starch (Contramid™; CA Patent No. 2,041,774 [Mateescu et al., Apr. 19, 1994]; U.S. Pat. No. 5,456,921 [Mateescu et al., Oct. 10, 1995]) and some of its derivatives (carboxymethyl cross-linked high amylose starch (CM-HASCL) and aminoethyl cross-linked high amylose starch (AE-HASCL); [U.S. Pat. No. 6,419,957 (Lenaerts et al., Jul. 16, 2002)]) allow the controlled release of drugs over 18-24 h.

For many bioactive agents (e.g., vaccines, probiotic microorganisms, therapeutic digestive enzymes, nutraceuticals and certain drugs), delivery inside a specific absorption window is optimal, rather than slow release throughout its passage through the GIT. For example, in certain instances it is therapeutically undesirable that an agent is delivered in the stomach whereas intestinal delivery is therapeutically desirable. Such a delivery requirement may for example be desired in cases where the agent is digested or degraded in the environment of the stomach or where the agent may act as a stomach irritant (e.g. aspirin) or induce nausea or vomiting.

As such, there is a continued need for an effective delivery system that can fulfil the desired controlled release profile according to particular therapeutic delivery requirements.

### SUMMARY OF THE INVENTION

The invention relates to polymeric material, compositions comprising such material, and uses thereof. The invention also relates to methods of preparing such material and compositions.

According to an aspect of the present invention, there is provided a composition comprising an uncrosslinked starch modified by an acidic group; and an agent, wherein the composition is resistant or substantially resistant to degradation in a first environment and is capable of degradation in a second environment. In an embodiment, the pKa of the acidic group of the composition is higher than the pH of the first environment and less than or equal to the pH of the second environment.

In embodiments, the pH of the first environment is less than or equal to about 5.0, in a further embodiment, from about 1.0 to about 5.0, in a further embodiment, from about 1.2 to about 4.5. In an embodiment, the first environment is the upper gastrointestinal tract (e.g. stomach) of an animal.

In an embodiment, the pH of the second environment is greater than about pH 5.0, in further embodiments, greater than about pH 5.5, 5.8, 6.0 or 7.2. In another embodiment, the pH of the second environment is from about 5.5 to about 8.0, or from about 5.8 to about 8.0. The second environment can be located in the lower gastrointestinal tract (e.g. intestine, e.g., small intestine) of an animal.

In an embodiment, the animal is a mammal, in a further embodiment, a human.

The starch of the composition described herein can, in an embodiment, be a high amylose starch, which in an embodiment comprises more than about 70% amylose. In another embodiment, the starch comprises less than or equal to about 70% amylose. In yet another embodiment, the starch comprises from about 30% to about 70% amylose. In embodiments, the origin of the starch of the composition is selected from the group consisting of corn, wheat, bean, pea, rice, potato, cereal, root or tuber.

In embodiments, the acidic group of the uncrosslinked starch of the composition described herein can either be a carboxyl, sulphate or a phosphate group. In an embodiment, the carboxyl group is a succinyl or a carboxyalkyl group. In a further embodiment, the alkyl is a lower alkyl. In yet a further embodiment, the lower alkyl is a C₁-C₆ alkyl. In still a further embodiment, the C₁-C₆ alkyl is a methyl group and the acidic group of the uncrosslinked starch is a carboxymethyl group.

In embodiments, the degree of substitution of starch with the acidic group described herein may vary. In an embodiment, the degree of substitution is greater than or equal to about 0.1 mmol/g. In another embodiment, the degree of substitution is from about 0.1 mmol/g to about 4.0 mmol/g. In still another embodiment, the degree of substitution is from about 0.1 mmol/g to about 1.5 mmol/g. In a yet another embodiment, the degree of substitution of the uncrosslinked starch is from about 0.1 mmol/g to about 1.25 mmol/g. In a further embodiment, the degree of substitution is from about 0.6 mmol/g to about 4.0 mmol/g. In yet a further embodiment, the degree of substitution is from about 0.6 mmol/g to about 1.5 mmol/g. In still a further embodiment, the degree of substitution is from about 0.6 mmol/g or to about 1.25 mmol/g. In yet a further embodiment, the degree of substitution is from about 0.6 mmol/g or to about 0.8 mmol/g.

The composition also comprises an agent. In embodiments, this agent may be a drug, a polypeptide, an enzyme, an organelle, a microorganism or a probiotic. In an embodiment, the drug is a small molecule. In another embodiment, the enzyme is a therapeutic enzyme, such as a digestive enzyme, such as a pancreatic enzyme, such as α-amylase or trypsin. In still another embodiment, the microorganism is a prokaryote, such as a bacterium. In embodiments the bacterium may be either gram negative or gram positive. In an embodiment, the bacteria may be Escherichia coli or Lactobacillus sp. In embodiments, the microorganism (e.g. bacterium) may be one conducive to reside in or that typically resides in the gastrointestinal tract. In embodiments, the microorganism (e.g. bacteria) may be a probiotic microorganism. In an embodiment, the bacterium may be a lactic acid bacteria or E. coli.

In embodiments, the composition may be formulated in an oral dosage form or unit. In an embodiment, the oral dosage form or unit may be a capsule, tablet, bead or a microsphere.

The agent described herein can also be in admixture with the uncrosslinked starch. In another embodiment, the agent can be substantially uniformly distributed throughout the composition.

In an embodiment, the composition described herein can also comprise a core portion comprising the agent and a coat portion substantially covering the core portion wherein the coat portion comprises the uncrosslinked starch modified by an acidic group. In an embodiment, the core portion further comprises a pharmaceutically acceptable excipient. In another embodiment, the core portion further comprises an uncrosslinked starch modified with an acidic group. In yet another embodiment, the degree of substitution of the uncrosslinked starch present in the coat portion is higher than the degree of substitution of the uncrosslinked starch present in the core portion.

According to another aspect, the invention also provides a commercial package comprising an uncrosslinked starch modified by an acidic group and instructions for preparing the composition described above. In an embodiment, the instructions set forth a method to obtain the composition. In another embodiment, the method set forth in the instructions comprises providing an agent and combining the agent with the uncrosslinked starch modified by an acidic group.

According to a further aspect, the invention further provides a method of preparing a composition for selective release of an agent in a target environment. The method comprises providing an uncrosslinked starch modified with an acidic group, providing an agent and combining the uncrosslinked starch with the agent, wherein, the composition is resistant or substantially resistant to degradation in a non-target environment and is capable of degradation in the target environment. In an embodiment, the pKa of the acidic group of the uncrosslinked starch is higher than the pH of the non-target environment and less than or equal to the pH of the target environment. In another embodiment, the method also comprises preparing uncrosslinked starch modified with an acidic group by modifying an uncrosslinked starch with an acidic group. In a further embodiment, the modification step comprises reacting the uncrosslinked starch with a haloalkyl-substituted carboxylic acid or with an anhydride. In still a further embodiment, the anhydride is succinic anhydride and the haloalkyl-substituted carboxylic acid is selected from the group consisting of monochloroacetic acid, 1-chloropropionic acid, 2-chloropropionic acid and chlorobutiric acid.

According to yet another aspect of the invention, there is provided a composition prepared according to the method described herein.

According to yet another aspect of the invention, there is provided a method for the selective delivery of an agent to a target environment, comprising introducing the composition described above into the target environment, e.g. by introducing the composition into a system comprising the target environment and allowing it to localize to the target environment.

According to a further aspect of the invention, there is provided a method for the selective delivery of an agent to a target environment. In an embodiment, the method comprises providing the above-noted composition (e.g. by preparing a composition according to the method described above) and introducing the composition into the target environment. In an embodiment, the target environment is the lower gastrointestinal tract or the small intestine of an animal. In an embodiment, the animal is a mammal or a human. In another embodiment, the agent is administered orally. In a further embodiment, the agent can be a drug, a polypeptide, an organelle, an enzyme or a microorganism.

According to still another aspect of the invention, there is also provided a commercial package comprising the composition described herein and instructions for administering the composition to an animal. In an embodiment, the instructions specify an oral administration of the composition to an animal. In another embodiment, the target environment for release of the agent in the composition is the lower gastrointestinal tract or the small intestine of an animal. In an embodiment, the animal is a mammal or a human.

According to yet another aspect of the present invention, there is provided use of the composition described herein to administer an agent to an animal.

According to a further aspect of the present invention, there is provided use of the composition described herein as a food additive.

According to yet a further aspect of the present invention, there is provided use of an uncrosslinked starch modified by an acidic group for the selective delivery of an agent to an environment. In an embodiment, the pH of the environment is higher than the pKa of the acidic group of the uncrosslinked starch. In another embodiment, the target environment is the lower gastrointestinal tract or the small intestine of an animal. In an embodiment, the animal is a mammal (e.g. a human).

According to yet a further aspect of the present invention, there is provided a composition comprising an uncrosslinked starch modified by an acidic group and a microorganism. In an embodiment, the acidic group of the uncrosslinked starch can be a carboxyl, sulphate or a phosphate group. In a further embodiment, the degree of substitution of the uncrosslinked starch with the acidic group can be from about 0.6 mmol/g to about 0.8 mmol/g. In still a further embodiment, the degree of substitution of the uncrosslinked starch with the acidic group is about 0.68 mmol/g. In another embodiment, the microorganism of the composition is a prokaryote (e.g. a bacterium). In a further embodiment, the microorganism is lyophilized.

According to still a further aspect of the present invention, there is provided a method for preserving viability of a microorganism, the method comprising combining the microorganism with an uncrosslinked starch modified by an acidic group. In an embodiment, the acidic group of the uncrosslinked starch can be a carboxyl, sulphate or a phosphate group. In a further embodiment, the degree of substitution of the uncrosslinked starch with the acidic group can be from about 0.6 mmol/g to about 0:8 mmol/g. In another embodiment, the microorganism is a prokaryote. In a further embodiment, the microorganism of the method is a bacterium. In another embodiment, the microorganism is lyophilized.

According to another a further aspect of the present invention, there is provided a commercial package comprising: an uncrosslinked starch modified by an acidic group and instructions for preserving viability of a microorganism. In an embodiment, the instructions set forth a method to prepare a composition, such as providing the microorganism and combining the microorganism with the uncrosslinked starch. In another embodiment, the microorganism of the commercial package is lyophilized. In a further embodiment, the microorganism of the commercial package is a prokaryote. In still a further embodiment, the microorganism of the commercial package is a bacterium.

According to yet another aspect of the present invention, there is provided use of an uncrosslinked starch modified by an acidic group for preserving viability of a microorganism.

### BRIEF DESCRIPTION OF THE DRAWINGS

FIG. 1. Evaluation of pH stability of tablets incubated in simulated gastric fluid (SGF). Tablets based on non-substituted (S-0) and substituted CM-S1, CM-S2 and CM-S3 polymers containing 4-nitrophenol as a pH indicator were incubated in pepsin-free SGF. Presence of the yellow colour, the intensity of which is indicated in Table II below, indicates pH stability. Treatments: (a) untreated tablets, (b) 5 min in distilled water, (c) 2 h in pepsin-free SGF—complete tablets and (d) 2 h in pepsin-free SGF—cross-sections of the tablets.

FIG. 2. Evaluation of the kinetics of pancreatin (α-amylase) activity in tablets based on S-0 and CM-S derivatives. The tablets were incubated in a simulated intestinal fluid (SIF) medium (37° C. and 50 rpm) and maltose liberation was measured (Mean ±S.D., n=3).

FIG. 3. Evaluation of the viability of bacteria formulated in tablets with S-0 and CM-S derivatives following incubation in an acidic medium. The tablets were incubated in pepsin-free SGF (37° C. and 50 rpm) and colony-forming units (CFU) were measured (Mean ±S.D., n=3).

FIG. 4. Evaluation of the release of viable bacteria formulated in tablets with S-0 and CM-derivatives following incubation in gastric and intestinal medium. The tablets were incubated in pepsin-free SGF for 1 h followed by 5 h in SIF (37° C. and 50 rpm) and colony-forming units (CFU) were measured (Mean ±S.D., n=3).

FIG. 5. Evaluation of the stability at 4° C. of unformulated E. coli compared to formulated E. coli based on CM-S2 or on S-0 derivatives. The stability tests were performed in 50 mL of pancreatic-free SIF (pH 6.8) at room temperature (Mean ±S.D., n 3).

FIG. 6. Evaluation of the stability of Lactobacillus rhamnosus bacteria formulated with CM-Starch in simulated gastric fluid. The tablets were incubated in 50 mL of SGF containing pepsin (37° C. and 50 rpm) and colony-forming units (CFU) were measured (n=2).

FIG. 7. Evaluation of the release of live Lactobacillus rhamnosus bacteria formulated with CM-Starch in simulated gastric and intestinal fluids. The tablets were incubated in SGF containing pepsin for 1 h followed by 8 h in SIF at 37° C. and 50 rpm and colony forming units (CFU) were measured (n=3).

FIG. 8. Evaluation of the stability of α-amylase formulated with CM-Starch or S-Starch in simulated gastric fluid. The tablets (200 mg) were incubated in 50 mL of SGF containing pepsin (37° C. and 50 rpm) and α-amylase enzymatic activity was measured at pH 7.2 (n=3).

FIG. 9. Evaluation of the loading of α-amylase formulated with CM-Starch or S-Starch. The tablets (200 mg) were incubated for one hour in 50 mL of SGF containing pepsin (37° C. and 50 rpm) and α-amylase enzymatic activity was measured at pH 7.2 (n=1).

FIG. 10. Evaluation of the liberation of α-amylase formulated with CM-Starch or S-Starch in pH 7.2 solution. The tablets (200 mg) were incubated for 1 h in 50 mL of SGF containing pepsin (37° C. and 50 rpm) and liberation of α-amylase at pH 7.2 at 37° C. and 50 rpm, was quantified from its enzymatic activity (n=4).

FIG. 11. Evaluation of the stability of trypsin formulated with CM-Starch or S-Starch in simulated gastric fluid. The tablets (200 mg) were incubated in 50 mL of SGF containing pepsin (37° C. and 50 rpm) and trypsin enzymatic activity was measured at pH 7.2 (n=5).

FIG. 12. Evaluation of the liberation of trypsin formulated with CM-Starch or S-Starch in pH 7.2 solution. The tablets (200 mg) were incubated for 1 h in 50 mL of SGF containing pepsin (37° C. and 50 rpm) and liberation of trypsin at pH 7.2 at 37° C. and 50 rpm, was quantified from its enzymatic activity (n=4).

### DETAILED DESCRIPTION OF THE INVENTION

The invention relates to a composition and its use for controlled delivery of an agent.

In an embodiment, the results described herein relate to studies of compositions which, once ingested, specifically deliver active agents in the lower gastrointestinal tractus.

When dried as carboxylate salts, carboxylic polymers, (such as, for example, alginate, carboxymethyl-cellulose and CM-HASCL) can be used for the preparation of compositions and formulations with bioactive agents which are particularly susceptible to alteration during the gastric passage. However, although the swelling of those polymers is fast, the dissolution of the matrix structure is incomplete and hence the matrix captures a proportion of the agent.

The studies described herein demonstrate that a non-crosslinked starch modified with an acidic group (e.g. non-crosslinked carboxymethyl-starch [CM-S] or non-crosslinked succinyl starch [S-Starch]) can serve as an excipient or carrier for the selective delivery of agents to a target region of interest, such as the lower gastrointestinal tractus.

The results presented herein show that CM-S and S-Starch based compositions may be prepared which are non-swollen and compact in the gastric environment and allow the release of the formulated agent in the intestinal environment. It is believed that the acid-modified (e.g. CM-S or S-Starch) polymer buffers the matrix preventing the release of the agent in the gastric environment. The CM-S or S-Starch polymer also allows dissolution and erosion of the composition in the intestinal environment. This erosion can further be accelerated by enzymatic hydrolysis with duodenal enzymes.

The swelling properties of ionic polymers, such as CM-S or S-Starch, depend on the pH and the ionic strength of medium (Mulhbacher et al., 2001). The swelling volume of polymers substituted with acidic groups increases with increasing pH values whereas the swelling volume of polymer substituted with basic groups decreases at increasing pH. With respect to ionic strength, the swelling volume of an acidic or basic polymer will decrease with increasing ionic strength.

The use of polymers modified by an acidic group, such as a carboxyl group, in the preparation of compositions and formulations, may, in embodiments, provide advantages such as:
a) carboxylate salt (e.g. sodium carboxylate) moieties act as buffers, thereby protecting the contents of the composition against the gastric acidic pH;
b) the shape of the form of the composition (e.g. tablet) is reduced in the acidic pH of the stomach thereby facilitating gastric passage; and
c) release of the agent in the lower gastrointestinal tractus is facilitated by the swelling of the composition at intestinal pH.

A role for polymers as pharmaceutical excipients and carriers is to protect the active agent against the acidic medium of the stomach and to deliver the agent to the intestinal mucosal site (Edelman et al., 1993). There is a wide range of polymers available for pharmaceutical use. Polymeric matrices based on polysaccharides (e.g. starch) are of interest in drug delivery.

As an example, high amylose starch is largely used in pharmaceutical industries as filler, binder or disintegrant (Roper, 1996). It contains more than 70% amylose (a non-ramified (1,4)-α-polysaccharide) and less than 30% amylopectin (branched with multiple side chains). The hydroxyl groups play an important role in the organization of the matrix network, which is an important factor in the control of the release of the formulated agent (Dumoulin et al., 1998; Ispas-Szabo et al., 2000).

There are many chemical modifications that can be done by partial substitution of hydroxylic groups of starch with various agents, such as haloalkyl-substituted carboxylic acid or an anhydride, leading to the formation of carboxyl groups.

It is described herein that polymeric carriers exhibiting carboxyl functions as salts (carboxylates), would exchange the cation for a proton in acidic (gastric) media, leading to a compact structure and providing a local buffer in the relative proximity of surface surroundings. This local buffer thus protects the carried active agent against acidic denaturation. When placed in a more neutral or weak alkaline environment, the protonated form will exchange the protons for cations, facilitating hydration and swelling. This causes, in return, the dissolution and erosion of the polymeric material, thereby releasing the agent. In further embodiments, ionization, protonation, solubilisation and/or enzymatic (Kost et Shefer, 1990) degradation of the polymers may also contribute to the chemical erosion mechanisms of the polymeric material.

Based on the studies described herein, uncrosslinked starch modified by an acidic group can thus be advantageously used in compositions for the specific delivery of agents to the lower gastrointestinal tractus (e.g. small intestine). In an embodiment, compositions comprising the uncrosslinked starch can be advantageously used in compositions for release of an agent in a specific manner, i.e. which may not commence until the agent has reached the lower GI tract (e.g., commencing at least about 1 hr following ingestion), together with rapid release once the target environment (e.g. lower GI tract) has been reached (e.g., over a period of 2-5 hrs once in the environment, or over a period of about 3-6 hrs following ingestion).

Uncrosslinked starch such as CM-S differs markedly from crosslinked starch such as CM-HASCL (such as the one derived from Contramid™), with respect to various parameters. Examples of such differences are summarised in Table I.

TABLE I
Examples of differences between CM-S and CM-HASCL
(obtained from Contramid ™)

| | CM-S | CM-HASCL |
|---|---|---|
| Cross-linking | No | Yes |
| Type of release | Specific to the | Over long intervals |
| | intestinal | through GIT (18-24 |
| | environment | hrs) |
| Mechanism of action | Swelling that occurs | Matrix stabilization |
| for the release of | with the passage | by cross-linking and |
| the agent | from the gastric to | hydrogen association |
| | the intestinal | to form a gel |
| | environment that | barrier which |
| | favours rapid | controls water |
| | dissolution of the | access inside the |
| | composition. Can be | matrix, hence |
| | further enhanced by | dissolution of the |
| | enzymatic hydrolysis | agent. |
| | *. | |
| Type of starch that | High amylose as well | High amylose starch |
| can be used | as regular (non-high | only |
| | amylose) starch | |
| Dissolution faster | Yes | No, matrix fragments |
| at pH 7.2 | | still present |

* It is likely that the matrix degradation may be accelerated by the duodenal alpha-amylase, for which CM-S presents a higher susceptibility as a substrate than CM-HASCL. It is also envisioned that CM-S will dissolute faster than alginate and carboxymethyl-cellulose, which are not recognized as substrates by the duodenal alpha-amylase.

The invention relates to an uncrosslinked modified starch and compositions thereof with an agent. The composition is substantially resistant to degradation in a first environment wherein there is no or substantially no release of the agent, and the composition is capable of degradation in a second environment wherein there is release of the agent. In embodiments, the first environment and the second environment correspond to the upper and lower gastrointestinal tract, respectively, of an animal. In further embodiments, the first environment and the second environment respectively refer to the stomach and the small intestine of an animal. In an embodiment, the animal is a mammal, in a further embodiment, a human.

In an embodiment, the release of the agent may be accomplished by transferring the composition from the first environment to the second environment, e.g. from the stomach to the small intestine. In a further embodiment, release of the agent may be accomplished by increasing the pH of the environment such that it surpasses the pKa of the uncrosslinked starch modified by an acidic group, thereby converting the first environment to the second environment.

“Uncrosslinked starch” as used herein refers to starch that has not been subjected to cross-linking via reaction with an exogenous crosslinking agent, i.e. that no exogenous cross-linking agent has been added to the starch prior to its use.

“Uncrosslinked starch modified with an acidic group”, also referred to herein as “USAG”, refers to any uncrosslinked starch as defined above which has been modified or substituted at any position with a moiety that confers an acidic function. In an embodiment, such an acidic function may be conferred by the attachment of a carboxyl moiety to the uncrosslinked starch.

“Degradation” or “degrade(s)” as used herein refers to the dissolution, decomposition, erosion, breakdown or otherwise destruction of or decrease in the integrity of the composition. In the context of a composition comprising an agent, degradation ultimately results in the release of the agent to the environment.

“Gastrointestinal tract” or GIT as used herein refers to the tube or passageway (that extends from the mouth to the rectum), where food is processed. The gastrointestinal tract is also known as the alimentary canal or digestive tract. The upper gastrointestinal tract refers to the alimentary tract from the mouth to the stomach. The lower gastrointestinal tract refers to the alimentary tract after the stomach to the rectum.

The compositions described herein also comprise an agent. The agent may in an embodiment be susceptible to gastric denaturation. In an embodiment, it is desirable that the agent be delivered to the lower GIT, e.g. the small intestine, due to for example susceptibility to denaturation in the stomach, improved or desired absorption in the lower GIT, or both.

An “agent” as used herein refers to any molecule of interest which is to be introduced into a target environment of interest. In an embodiment, the agent may represent a bioactive molecule for oral administration to a subject. Various agents can be used such as drugs (e.g., small molecules, larger molecules and complexes, salts thereof, nutritional supplements) polypeptides (e.g., native, isolated or fragments), polynucleotides (e.g., DNA, RNA or both), extracts (e.g., from plants, microorganisms, virus, animals, cells), fat (e.g., lipids, oils, fatty acids), organelles, microorganisms (e.g., eukaryotes such as fungi, prokaryotes such as bacterium, and viruses) and probiotics. The agent may in embodiments comprise a bioactive molecule such as a protein or enzyme. The agent may represent an active molecule or may be for example an inactive molecule which requires activation at or before reaching the site of action, such as a prodrug.

“Probiotics” refers to materials comprising microbial cells which transit the gastrointestinal tract and which, in doing so, benefit the health of the consumer (Tannock et al. 2000). As such, “probiotic cultures” or “probiotic cells” or “probiotic microorganisms” as used herein refers to microbial cells or material comprising microbial cells which may be introduced into the gastrointestinal tract of an animal, and may reside in/transit the gastrointestinal tract and may provide some functional effect on the physiology/activity thereof, such as a functional effect to benefit the health of the animal. In an embodiment, the animal is a mammal, in a further embodiment, a human.

In embodiments, the starch used in the compositions described herein may be derived from high-amylose starch, regular starch, or mixtures thereof. Starch contains two principal components: amylose and amylopectin. Amylose or high amylose starch typically contains more than about 70% amylose and less than about 30% amylopectin; whereas regular starch (non-high amylose) usually contains from about 30% to 70% amylose. In further embodiments, starch can be obtained from sources such as corn, wheat, bean, pea, rice, potato, cereal, root and tuber starch.

In an embodiment, the uncrosslinked starch is modified with an acidic group. In an embodiment, the modification occurs at a hydroxyl group on the starch. In embodiments, the added acidic group may be a carboxyl, sulfatidyl or phosphatidyl group, or combinations thereof. In the case of modification with a carboxyl group, the starch may in embodiments be reacted with a haloalkyl-substituted carboxylic acid, such as monochloroacetic acid, 1-chloropropionic acid, 2-chloropropionic acid, chlorobutyric acid or with an anhydride such as succinic anhydride. The number of acidic groups attached to the starch, or the degree of substitution, may vary according to further embodiments. In embodiments, depending on the specific requirements or uses of the composition and other components present in the composition (such as the active agent) for any given application, the degree of substitution may be greater than or equal to about 0.1 mmol/g. In another embodiment, the degree of substitution is from about 0.1 mmol/g to about 4.0 mmol/g. In still another embodiment, the degree of substitution is from about 0.1 mmol/g to about 1.5 mmol/g. In a yet another embodiment, the degree of substitution of the uncrosslinked starch is from about 0.1 mmol/g to about 1.25 mmol/g. In a further embodiment, the degree of substitution is from about 0.6 mmol/g to about 4.0 mmol/g. In yet a further embodiment, the degree of substitution is from about 0.6 mmol/g to about 1.5 mmol/g. In still a further embodiment, the degree of substitution is from about 0.6 mmol/g or to about 1.25 mmol/g. In yet a further embodiment, the degree of substitution is from about 0.6 mmol/g or to about 0.8 mmol/g. In still a further embodiment, the degree of substitution is about 0.68 mmol/g.

“Acidic group”, as used herein, refers to a group which may gain a proton in an environment having a pH lower than its pKa and loses a proton in an environment having a pH greater than its pKa. In an embodiment, the loss of the proton results in the creation of a negatively charged group which can associate with a cation to form a salt, such as in the case of a carboxyl function where loss of a proton results in a carboxylate which can form a carboxylate salt. Decreasing the pH of the environment or transferring the carboxylate-containing starch to a lower pH environment, i.e. to levels below the pKa, shall result in protonation of the carboxylate to a carboxylic acid, and, in the case of a carboxylate salt, displacement of the cation with a proton.

The degree of substitution of a modified starch can be measured in various ways. In the case of an acidic modification, the degree of substitution may be measured by titration of the acidic group with a base. In an embodiment, the degree of substitution is determined by potentiometric titration of the (e.g. carboxymethyl groups) and is expressed in mmol of functional groups per g of polymeric powder (mmol/g).

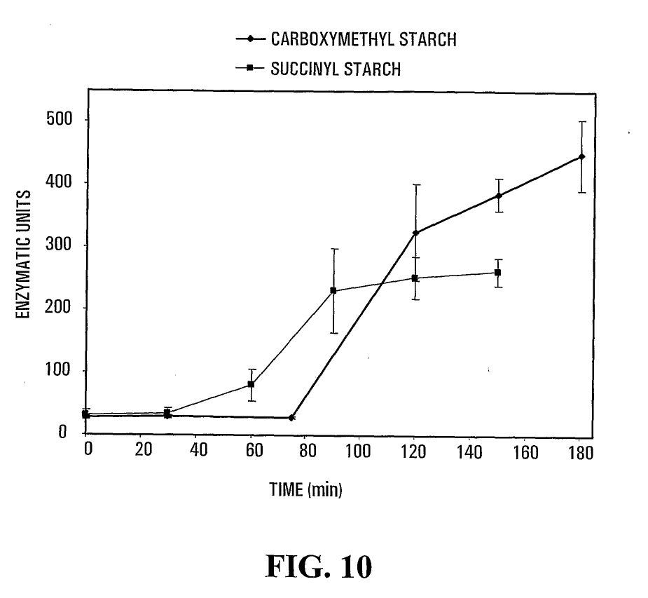

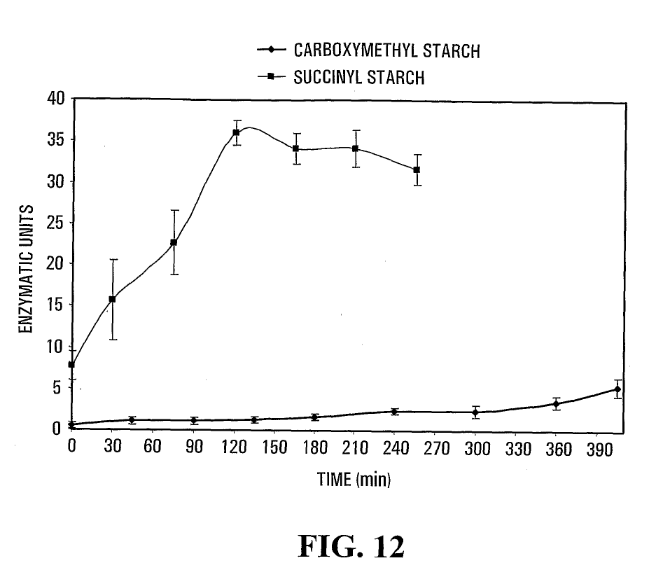

The USAG to be used in the composition may be designed for a particular application based on various parameters. For example, the degree of substitution, as shown herein, confers different properties on the composition, notably with respect to the release of the agent. Therefore, the degree of substitution is a further parameter, which may be varied to design a USAG for a particular application. For example, an increased degree of substitution appears to result in greater stability of the composition in the lower pH environment, i.e. the first-environment noted above where release of the agent is not desired. Varying the degree of substitution may also result in different release properties of the composition in the second environment, i.e. that where release is desired. Further, the degree of substitution may be varied to be more conducive to particular types of agents, such as using a USAG with a higher degree of substitution for a small molecule. Moreover, the release properties of the composition may be controlled not only by degree of substitution, but also by the choice of substituent. For example, use of a succinyl group as substituent resulted in an increased rate of release over carboxymethyl (FIGS. 10 and 12).

The pKa of the USAG plays a role in controlling the release of the agent from the composition, as no or substantially no release shall occur in a first environment having a pH lower than the pKa, and release shall occur in a second environment having a pH higher than the pKa. In embodiments, the first and second environments represent the upper and lower gastrointestinal tracts, respectively, e.g. the stomach and small intestine, respectively. Thus, to design a USAG to minimize release in the stomach (which has a pH of about 1.2 to about 4.5) and allow release in the small intestine (which has a pH of about 6.4 to about 8.0), it would be appropriate to design the USAG to have a pKa greater than about 4.5, and, in an embodiment, not greater than 6.4. For example, carboxymethyl uncrosslinked starch, which has a pKa of about 5.8, could be used in such a case. As mentioned above, other acidic substitutions (e.g., phosphate and sulphate) of the USAG can be also be used for intestinal delivery. The pKa may be varied, however, depending on the selectivity desired for any particular use. Increasing the pKa of a USAG shall result in an increase in the pH required for the environment where release of the agent is desirable. Similarly, decreasing the pKa of the USAG shall result in a decrease of the pH required for release to occur. The pKa of the USAG may be varied by the choice of the acid modification used, as well as by combining different types of acid modifications. By varying this parameter, an USAG may be designed for any particular system where delivery is not desired in a first environment but desired in a second environment, whereby the pH of the second environment is higher than that of the first environment.

The compositions of the present invention can also be formulated in a dosage form or unit, in an embodiment an oral dosage form or unit. In an embodiment, the dosage form or unit may be a capsule, tablet, bead or a microsphere. Therapeutic compositions typically should be sterile and stable under the conditions of manufacture and storage. In an embodiment the composition can be formulated as an ordered structure suitable to high agent concentration.

In an embodiment, the composition further comprises a pharmaceutically acceptable carrier or excipient.

In another embodiment, the agent is incorporated in the composition with the uncrosslinked starch in such a way that it is incorporated substantially throughout the composition, i.e. in a substantially uniform distribution or mixture with the uncrosslinked starch in the composition. In a further embodiment, the composition may be structured so as to comprise an inner or core portion and an outer or coat portion. In an embodiment, the coat covers part of the core. In a further embodiment, the coat covers substantially all of the core. In yet a further embodiment, the coat covers all of the core.

In an embodiment, the core comprises the agent. In an embodiment, the coat comprises the USAG. In a further embodiment the coat may comprise both the agent and the USAG.

In a further embodiment, the core may comprise both the agent and the USAG. In an embodiment, either the core portion or the coat portion or both comprises or further comprises a pharmaceutically acceptable excipient. In the case where both the coat and the core comprise USAG, the degree of substitution of the USAG present in the coat is in an embodiment higher than the degree of substitution of the USAG present in the core.

For the core portion of the compositions described herein, other pharmaceutical polymeric excipients can be used. These polymers can include, but are not limited to polymeric matrices based on polyvinylacetate [PVAc], polyvinylalcohol [PVA], polyvinylpyrollidone [PVP], acrylic polymers (i.e. poly(hydroxyethyl)methacrylate) [PHEMA] or polysaccharide based on chitosan, alginate or cellulose derivatives (e.g. hydroxymethylpropyl cellulose [HPMC]), polylactic-glycolic acid (PLGA) and others such as polyanhydrides, polyglycolic acid, collagen, polyorthoesters, polylactic acid and polylactic, polyglycolic copolymers (PLG). Many methods for the preparation of such formulations are patented or generally known to those skilled in the art.

As used herein “pharmaceutically acceptable carrier” or “excipient” can also include any and all antibacterial and antifungal agents, isotonic and absorption delaying agents, and the like that are physiologically compatible. In one embodiment, the carrier is suitable for oral administration. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

The invention also relates to methods of preparing the composition, comprising providing a USAG and formulating the USAG with an agent. In an embodiment, the method further comprises the step of preparing the USAG by modifying an uncrosslinked starch with an acidic group, prior to formulating the USAG with the agent.

The invention also relates to kits or (commercial) packages that can be used for the preparation and/or use of the compositions described herein. For example, the invention provides a commercial package comprising an USAG together with instructions to formulate a composition for delivery of an agent to the above-mentioned second environment. The commercial package may further comprise instructions for delivery of the composition so formulated to the above-mentioned second environment. The invention further provides a commercial package comprising the composition together with instructions for delivery of an agent to the above-mentioned second environment.

The invention further relates to a method of administering an agent which comprises providing (in a further embodiment, preparing) the composition described herein and the introduction of the composition into a selected environment. Commercial packages comprising the compositions and instructions for administration are also contemplated.

The invention also relates to food additives comprising the composition herein described.

The invention further relates to various uses of the composition for preparing a medicament, a vaccine and food or nutritional supplement. Uses of the composition for selective delivery of an agent is also described.

The invention also relates to a composition for preserving the viability of a microorganism (e.g. a prokaryote, [e.g. bacterium]), comprising an uncrosslinked starch modified by an acidic group (USAG) and the microorganism. The invention further relates to a method for preserving the viability of a microorganism by combining the microorganism with an USAG. “Preserving viability” as used herein with respect to a composition comprising an USAG and a microorganism, refers to a smaller decrease in viability of the microorganism in the composition as compared to the decrease in viability observed in a corresponding microorganism which is not in such a composition, when stored for a similar period of time under comparable environmental conditions. Thus any observed decrease in viability over time, if at all, would be less for the microorganism in the composition than for the corresponding free microorganism.

The invention further provides a corresponding commercial package comprising the USAG together with instructions for combining the USAG with a microorganism (e.g. bacterium) in order to preserve its viability.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The following examples are illustrative of various aspects of the invention, and do not limit the broad aspects of the invention as disclosed herein.

### EXAMPLES

### Materials

High amylose corn starch (Hylon VII™) from National Starch; pancreatin (porcine pancreas) eight times strength (with α-amylase, lipase and proteolytic activities) from American Chemicals; agar powder USP from Anachemia Chemicals Ltd.; yeast extract from Difco Laboratories; 3,5-dinitrosalicylic acid and monochloroacetic acid from Aldrich; Pepsin from Sigma Chemical Co., MRS (DeMan, Rogosa and Sharpe, 1960) Lactobacilli powder from Difco Laboratories; Lactobacillus rhamnosus bacteria (strain HA-111, lyophilized) from Harmonium International Inc. The chemicals were used without further purification.

### Example 1

### Synthesis of Carboxymethyl Starch Derivatives (CM-S)

### Synthesis of Carboxymethyl Starch

The synthesis of polymeric derivatives were performed as described previously by Schell et al. (1978) with modifications. Three variants of non cross-linked carboxymethyl high amylose starch (CM-S) with different degrees of substitution: CM-S1, CM-S2 and CM-S3 were obtained.

Briefly, 70 g of high amylose starch was suspended in 170 mL of distilled water and warmed at 50° C. under continuous stirring in a Hobart planetary mixer. Then, 235 mL of 1.5 M NaOH was added slowly and the reaction medium was homogenized for 20 min at 50° C. The different degrees of substitution of the polymeric variants were obtained by adding different amounts of monochloroacetic acid dissolved in a minimal volume of distilled water to the alkaline reaction medium. Thus, 5 g of monochloroacetic acid was added for CM-S1 synthesis, 45.5 g for CM-S2 and 70 g for CM-S3 synthesis. The pH was maintained between 9-10 by adding small volumes, if necessary, of a 10 M NaOH solution to the alkaline suspension (55 mL for CM-S2 synthesis and 100 mL of 10 M NaOH for CM-S3 synthesis; none for CM-S1 synthesis). The reaction media were maintained under continuous stirring for 1 h at 50° C.

To end the substitution reaction, 130 mL of distilled water (50° C.) were added to the gel slurries and 250 mL-350 mL of acetic acid solution (20 mL of glacial acetic acid in 139 mL of distilled water and 244 mL of distilled water preheated at 50° C.) were added slowly to neutralize the stirred suspensions. The final pH of the suspensions was neutral to slightly acidic (6.8-7.0). The reaction media were cooled at the room temperature. The neutralized suspensions were then four times dissolved in minimum fresh water then precipitated again, as follows:

A volume of 600 mL of pure acetone was added slowly to each of the neutralized suspensions which were then stirred for 30 min at room temperature. The suspensions were subsequently filtered and gels that formed on the filter were recovered. Each gel was resuspended in 600 mL of acetone/water solution (60:40 v/v) and stirred for 30 min. The resuspended gels were once again filtered. These last two procedures (filtering+resuspension) were repeated twice for CM-S1 and once for CM-S2 and CM-S3. For CM-S1 synthesis, the gel recovered after the last filtration was resuspended in 1 L of acetone/water solution (80:20 v/v), stirred at room temperature for 30 min and then filtered.

Each gel recovered was resuspended in 600 mL of pure acetone and filtered. The last two operations (resuspension and filtering) were repeated twice to form the polymeric derivatives pastes. Finally the pastes were dried with acetone, blended and sieved to obtain particles smaller than 500 μm.

### Determination of the Degree of Substitution of the Polymeric Derivatives (CM-S)

The degree of substitution of the polymeric derivatives was determined by potentiometric titration of carboxymethyl (CM) groups with 0.2 N NaOH. The degree of substitution is expressed in mmol of functional groups per g of polymeric powder (mmol/g). The degree of substitution (DS) was determined as 0.142 mmol/g polymer for CM-S1 and 0.68 mmol/g polymer for CM-S2 and 1.25 mmoles/g for CM-S3 derivative.

Preparation of a Non-Derivatized High Amylose starch (S-0) Used as Control

A starch that has been treated with NaOH (1 h, 50° C.) but not with monochloroacetic acid was used as control in all experiments (referred to herein as S-0). The gel slurry was neutralized with acetic acid at the room temperature, thoroughly washed with acetone/water mixture (60:40 v/v) and finally dried with pure acetone.

### Example 2

### Determination of the Stability of Polymeric Derivatives (CM-S) and Control Polymer (S-0) in Tablet Form at Gastric and Intestinal pH

### Determination of the Stability of Polymeric Derivatives (CM-S) and Control Polymer (S-0) in Tablet Form at Gastric pH

The tablets comprising the S-0 and CM-S1, CM-S2 and CM-S3 polymers were obtained by direct compression of a mixture of a powder of the different polymers and 4-nitrophenol (which acts as pH indicator; 1-10 mg/tablet), at 2.5 T/cm² using a manual hydraulic press (Carver) and 9.0 mm cylinder outfits to get 200 mg tablets.

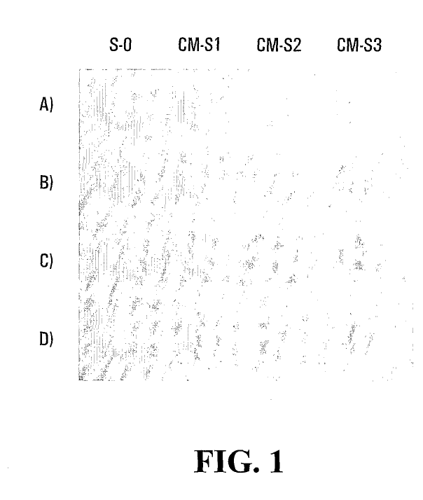

A pepsin-free simulated gastrointestinal fluid (SGF; pH 1.2) was prepared as described in U.S. Pharmacopeia (XXII). Briefly, 2.0 g of sodium chloride was dissolved in mL of concentrated hydrochloric acid. Water was added to complete the volume to 1000 mL. The tablets were incubated in 50 mL of pepsin-free simulated gastric fluid for 2 h at 37° C., agitated at 50 rpm. The disintegration of the tablets was monitored. The tablets were also were cross-sectioned and the modification of the pH indicator colour was visually evaluated. The table below summarizes the results of FIG. 1.

TABLE II
Qualitative evaluation of color density (yellow), of the tablets shown
in FIG. 1 (− = no yellow colour; + , ++, +++,
++++ = increasing density of yellow colour).

| | S-0 | CM-S1 | CM-S2 | CM-S3 |
|---|---|---|---|---|
| (a) | ++ | ++ | + | + |
| (b) | ++++ | ++++ | ++++ | ++++ |
| (c) | − | − | + | ++ |
| (d) | − | + | + | ++ |

The tablets based on carboxymethyl starch (CM-S1, CM-S2 and CM-S3) maintained their shape (tablets were almost 20 intact) in pepsin-free SGF after 2 h at 37° C. and 50 rpm, whereas the tablets based on the control starch (S-0) were disintegrated. For CM-S2 and especially for CM-S3, a gel layer formed at the surface of the tablets. The gel front seemed to progress toward the centre of the tablet. After 2 h in pepsin-free SGF, the entire CM-S3 tablet gelled but maintains its shape in solution.

For the control tablets containing S-0, it was observed that the disintegrated parts of the tablets were colourless indicating that S-0 did not stabilize the pH indicator formulated in the tablet. The CM-S3 tablet presented the highest pH protection followed by, in decreasing order of protection, CM-S2 and CM-S1 (FIG. 1 and Table II).

### Determination of Stability of Polymeric Derivatives (CM-S) and Non-Derivatized Polymer (S-0) in Tablet Form in the Presence of Pancreatin

The tablets comprising the S-0, CM-S1, CM-S2 and CM-S3 (described in Example 1) polymers were obtained by direct compression of a powder of the different polymers, at 2.5 T/cm² using a manual hydraulic press (Carver) and 9.0 mm cylinder outfits to get 200 mg tablets.

A simulated intestinal fluid was prepared according to U.S. Pharmacopeia [XXII]: 6.8 g monobasic potassium phosphate were dissolved in 750 mL distilled water, pH was adjusted to 7.5±0.1 with 0.2 M NaOH and volume was adjusted to 1 L with water. Each tablet (200 mg) was incubated in 50 mL of simulated intestinal fluid (SIF) and containing non-sterile pancreatin 1 USP at 37° C. and agitated at 50 rpm. The shape of the tablets was visually examined every hour over a 5 h period. Aliquots were sampled every hour to determine maltose liberation (an effect of pancreatin alpha-amylase enzymatic activity) with 1% 3,5-dinitrosalicylic acid (DNS) as described by Noelting et Bernfeld [1948]. Briefly, 1 mL of DNS reagent was added to 2 mL of each aliquot. The mixture was heated in a boiling bath for 5 min. The boiled samples were then rapidly placed in an ice-cold water bath to stop the reaction and diluted with 15 mL of distilled water. The absorbency was read at a wavelength of 535 nm. The liberated reducing sugars were calculated using a calibration curve of maltose. Each experiment was performed in triplicate.

The S-0 tablets were disintegrated after 1 h in SIF whereas the tablets based on substituted starch (CM-S1, CM-S2 and CM-S3) maintained their shape for more than 2 h. The CM-S1 tablets presented the capping phenomenon (a partial opening of the tablet leading to an increase of the release surface) after 3 h in SIF. The CM-S2 and CM-S3 tablets were partially solubilized after 4 h and 3 h, respectively (Table III). It was also observed that the swelling of the tablets increases with respect to the degree of substitution of the different polymer.

TABLE III
Visual evaluation of the stability of tablets based on polymeric CM-S
derivatives, in the simulated intestinal fluid medium containing pancreatin,
at 37° C. and 50 rpm (Disin.: disintegrated).

| Poly- | | | | | |
|---|---|---|---|---|---|
| mer | 1 h SIF | 2 h SIF | 3 h SIF | 4 h SIF | 5 h SIF |
| S-0 | Disin. | Disin. | Disin. | Disin. | Disin. |
| CM- | No visible | No visible | Capping; | Capping; | Capping; |
| S1 | dissolution; | dissolution; | moderate | moderate | moderate |
| | low swelling | moderate | swelling | swelling | swelling |
| | | swelling | | | |
| CM- | No visible | No visible | No visible | Partially | Dissolved |
| S2 | dissolution; | dissolution; | dissolution; | dissolved | |
| | moderate | moderate- | high | tablet | |
| | swelling | high | swelling | | |
| | | swelling | | | |
| CM- | No visible | No visible | Partially | Dissolved | Dissolved |
| S3 | dissolution/- | dissolution/- | dissolved | tablet | |
| | moderate- | high | | | |
| | high | swelling | | | |
| | swelling | | | | |

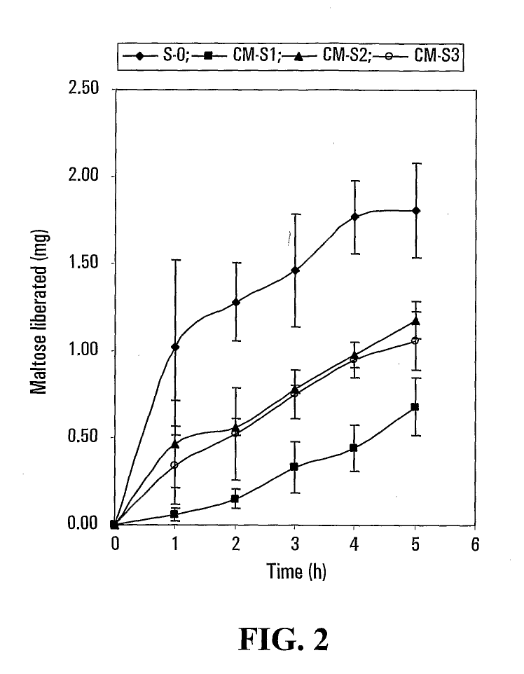

The CM-S tablets were found to be susceptible to hydrolytic erosion and disintegration by intestinal α-amylase, despite the fact that the starch is chemically modified (FIG. 2). CM-S1 tablet presented the highest stability to α-amylase activity when compared with the tablets from other CM-S. However, a slightly higher susceptibility (not significant) to amylolysis was observed for CM-S2 tablets when compared to CM-S3 tablets. All CM-S tablets were more resistant to amylolysis than S-0 tablet.

Swelling was less pronounced for CM-S1 tablets than for CM-S2 or CM-S3 tablets.

### Example 3

### Formulation of Lyophilized Microorganisms

Lyophilized non pathogenic bacteria Escherichia coli was formulated in tablet forms, with the S-0 and CM-S starch derivatives.

### Determination of Viability of Bacteria in Acidic Medium (In Vitro)

Tablets (200 mg) based on S-0, CM-S1, CM-S2, CM-S3 derivatives (described in Example 1) and containing 10 mg of lyophilized E. coli were formulated by direct compression at 2.5 T/cm². Tablets were placed individually in 50 mL of sterile pepsin-free SGF (pH 1.2) for different periods of time (30, 60, 90 and 120 min) at 37° C., 50 rpm (simulating the gastric passage) and their shape was examined visually. The tablets were then transferred into 50 mL of sterile pancreatic-free SIF (pH 6.8) and crushed. Aliquots of 1 mL were serially diluted (dilution factor between 10⁻¹ and 10⁻⁶). A volume of 100 μL of each dilution was plated on nutritive agar-agar (2% agar) in order to determine the number of bacterial colony forming unit (CFU). As a control, 10 mg of non-formulated bacteria was incubated in 50 mL of pepsin-free SGF (pH 1.2) for 30, 60, 90 and 120 min under the same conditions (37° C. and 50 rpm).

The S-0 tablets loaded with bacteria (E. coli) disintegrated during the first 30 minutes of incubation in pepsin-free SGF, whereas those based on the CM-S derivatives were not. After 2 h in the acidic medium, CM-S1 tablets presented a very low swelling, whereas those based on the other two substituted polymers show a low (CM-S2) and moderate-low swelling (CM-S3).

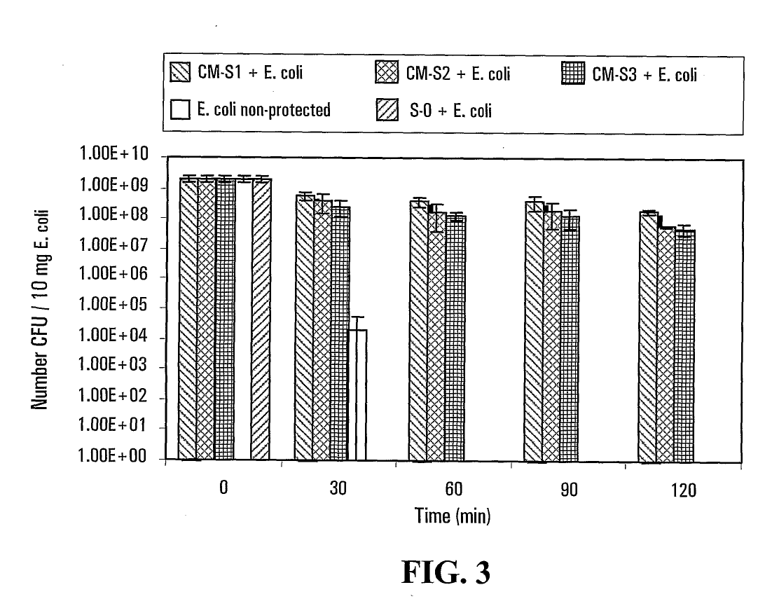

The bacterial viability tests demonstrated that the CM-S polymeric derivatives were able to protect microorganisms for 2 h against acidic denaturation (pepsin-free SGF, 50 rpm, 37° C.), whereas the S-0 was not (FIG. 3). After 30 min of acidic treatment, the viability of bacteria formulated with the CM-S derivatives was higher for all substituted polymers than the viability of the non-formulated control bacteria. No significant differences were noticed between the bacterial viability obtained after 60 and 90 min of incubation for the various CM polymers. After 2 h in pepsin-free SGF, all three substituted polymers provided bacterial protection from the acidic medium. The highest protection was obtained in CM-S1 tablet followed, in decreasing order of protection, by CM-S2 and CM-S3.

This assay showed that non-formulated bacteria persist in an acidic medium (pH 1.2) only for 30 min. Initially, 2.02×10⁹ CFU of non-formulated bacteria (10 mg) was placed in the SGF medium. After a 30 min incubation, only 2.00×10⁴ CFU remained from the non-formulated bacteria. No CFU were recorded after a 60 min incubation of the non-formulated bacteria in pepsin-free SGF. For the S-0 polymer, the tablets were disintegrated during the first 30 min of incubation in pepsin-free SGF and no viable bacteria were recuperated.

### Determination of Bacterial Delivery in Intestinal Medium (In Vitro)

Tablets (200 mg) based on S-0, CM-S1, CM-S2, CM-S3 (described in Example 1) and containing 10 mg of lyophilized E. coli were incubated in 50 mL of sterile pepsin-free SGF (pH 1.2) for 1 h at 37° C., under 50 rpm shaking and then transferred into 50 mL of sterile SIF containing pancreatin (pH 7.5±0.1), as specified in U.S. Pharmacopeia [XXII], and incubated for 5 h at 37° C. and 50 rpm.

The tablet shape was examined visually during the entire incubation period. Aliquots of 1 mL were taken after 1 h in pepsin-free SGF and every hour in the simulated intestinal medium to evaluate the viability of the bacteria (number of CFU) liberated from each tablets. A volume of 100 μL of each dilution was used for every ordinary nutritive agar-agar plate.

As a control for the acidic medium (SGF) on bacterial viability, 10 mg of lyophilized non-formulated bacteria was incubated for 1 h in 50 mL of sterile pepsin-free SGF (pH 1.2) at 37° C. and 50 rpm. Then, a sample of 1 mL was taken to evaluate the viability of the bacteria. As a control for the simulated intestinal medium on bacterial viability, 10 mg of lyophilized non-formulated bacteria was incubated for 5 h in 50 mL of sterile SIF containing pancreatin (pH 7.5±0.1) at 37° C. and 50 rpm. Samples of 1 mL were taken every hour to evaluate the bacterial viability. The initial amount of the E. coli in the preparation (number of CFU/10 mg lyophilized bacteria) was determined in sterile pancreatin-free SIF (pH 6.8) at room temperature. All the tests were performed in triplicate and the colonies were counted after aerobic incubation at 37° C. for 24 h.

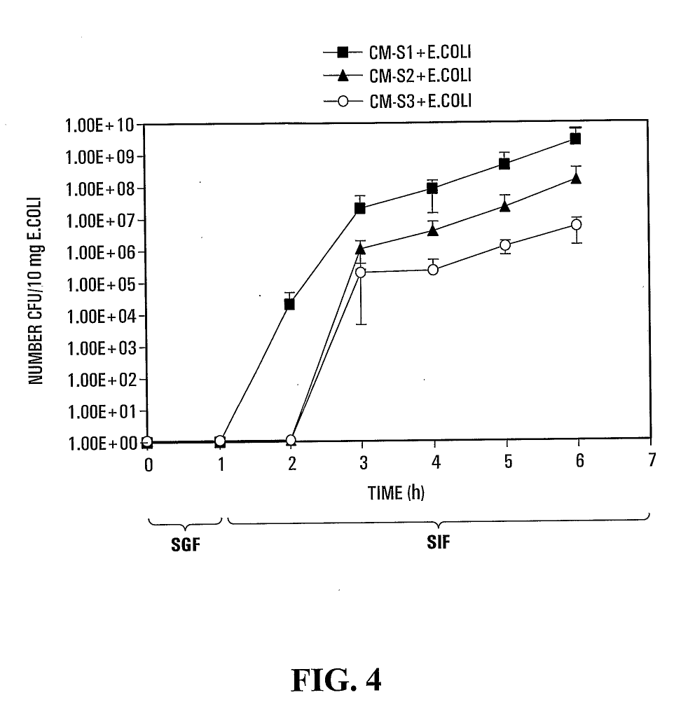

The CM-S2 and CM-S3 containing tablets were partially dissolved after 2-3 h in SIF (pH 7.5±0.1) at 37° C., 50 rpm, and release of the bacteria was observed (FIG. 4). The CM-S1 tablet presented a capping phenomenon after 1-3 h of incubation and released a higher amount of bacteria than CM-S2 and CM-S3 (FIG. 4). Initially, after 1 h in SGF, no viable bacteria were found in the gastric medium for the CM-substituted polymers and control (S-0). The CM-S1 tablets liberated 2.09×10⁴ CFU/10 mg bacteria during the first hour in SIF whereas CM-S2 and CM-S3 liberated no bacteria in this interval. After 2 h in SIF, the liberation from CM-S2 and CM-S3 is observed. It was found that an increasing degree of substitution resulted in a decrease liberation of viable bacteria. CM-S3, although found to afford best buffering properties (FIG. 1), is also the most hydrophilic derivative given the high swollen properties of the CM-S3 tablets. Thus, it is the most susceptible to the acidic attack and liberated only a small amount of viable bacteria in SIF. During the 5 h incubation period SIF, no colony forming units were found for the control polymer (S-0).

### Example 4

### Stability of E. coli Formulated with CM-S2 After 6 Months Storage Under Refrigeration

Tablets (200 mg) based on S-0 or CM-S2 derivatives (as described in Example 1) and containing 10 mg of lyophilized E. coli were formulated by direct compression at 2.5 T/cm². The tablets were incubated at 4° C. for 3 and 6 months. As a control, the bacteria were incubated in a tube in the same conditions.

After the incubation, the CM-S2 and S-0 formulations were transferred in 50 mL of sterile pancreatic-free SIF (pH 6.8) and rapidly crushed at the room temperature. Aliquots of 1 mL were serially diluted (dilution factor between 10⁻¹ and 10⁻⁶) and a volume of 100 μL of each dilution was used for each ordinary nutritive agar-agar plate in order to determine the number of bacterial colony forming unit (CFU). For the control (E. coli non-protected), the number of CFU was also determined in pancreatic-free SIF. The number of CFU/10 mg dry bacteria was determined at time 0 and after 3 or 6 months for each group of samples.

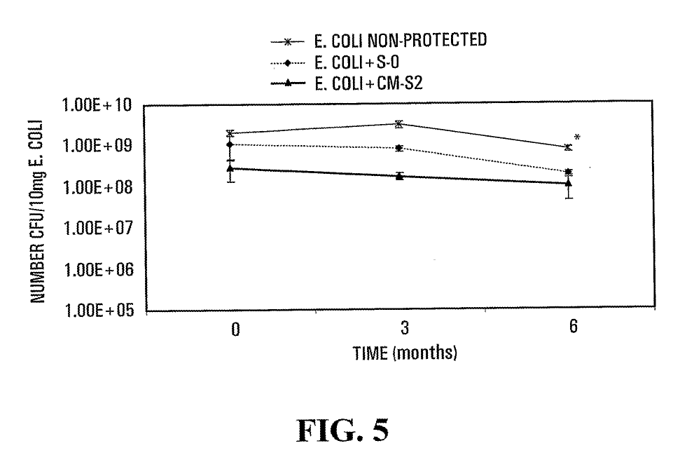

The count of viable bacteria formulated as tablets with CM-S2 and S-0 decreased slightly when stored for 6 months under refrigeration at 4° C. For the unprotected, free E. coli, a slight decrease of the bacterial viability was also obtained after six months of storage in similar conditions (FIG. 5).

### Example 5

### Formulation of Microorganism with CM-S Excipients

Determination of Viability of Lactobacillus rhamnosus Bacteria in the Simulated Gastric Medium

Tablets (200 mg) based on CM-S and containing 10 mg of lyophilized L. rhamnosus (approximately 10⁹ bacteria) were formulated by direct compression at 2.5 T/cm². The initial amount of L. rhamnosus in the lyophilized preparation (number of bacterial colony forming units (CFU)/10 mg lyophilized bacteria) was determined in sterile PBS (pH 7.4) at room temperature. The tablets were then placed individually in 50 mL of sterile simulated gastric fluid (SGF) pH 1.2 prepared as described in U.S. Pharmacopeia [XXIV] for different times at 37° C. (simulating the gastric passage), under agitation at 50 rpm, using the incubator shaker as above. Their shape was first examined visually. The viability of bacteria was evaluated after 60 and 120 min in SGF containing pepsin. After the appropriate period of incubation in SGF, the tablets were transferred into 50 mL of sterile PBS (pH 7.4), crushed and then aliquots of 1 mL were ten-fold serially diluted. A volume of 100 μL of each dilution was plated on a nutrient MRS Lactobacilli plate in order to determine the number of CFU. As control for the active agent, 10 mg of free (non-formulated) lyophilized bacteria was used in the same conditions. The tests were performed in duplicate and the colonies were counted after aerobic incubation at 37° C. for at least 48 h.

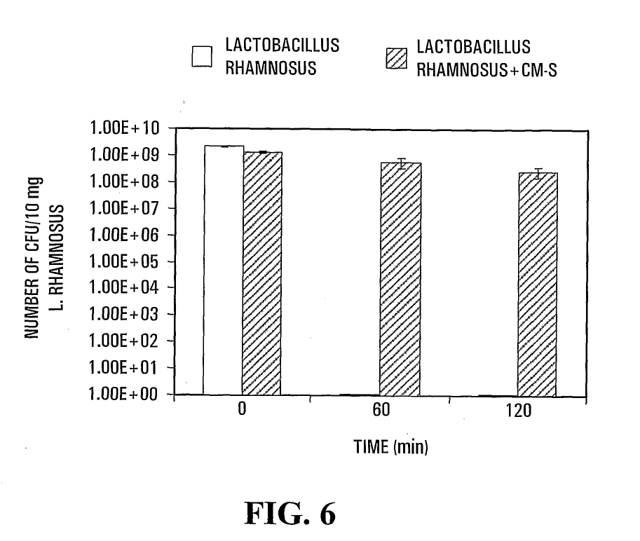

The CM-S tablets showed a moderately-low swelling after 2 h in SGF containing pepsin. The L. rhamnosus viability tests showed that, when formulated as tablets, the polymeric excipient was able to protect the bacteria against 120 min of acidic denaturation (FIG. 6). After this period, the number of viable bacteria formulated with CM-S was 5.73×10⁸ (60 min) and 2.7×10⁸ (120 in). For the free L. rhamnosus suspension, no viable bacteria were observed after 60 min and 120 min of incubation in acidic medium (pH 1.2). This assay also showed that non-formulated bacteria cannot persist in simulated gastric medium (pH 1.2) for 60 min or longer whereas the CM-S can protect the bacteria in SGF medium for 2 hours.

Determination of L. rhamnosus Delivery in the Simulated Intestinal Medium

The same formulations as above were incubated in 50 mL of sterile SGF (pH 1.2) for 1 h at 37° C., under agitation (50 rpm) and then transferred into 50 mL of simulated intestinal fluid (SIF) containing pancreatin prepared as described in U.S. Pharmacopeia [XXIV], and incubated for 8 h at 37° C. at 50 rpm. The tablet shapes were again examined visually during the entire incubation period. Samples of 1 mL were taken after 1 h in SGF and every hour in the SIF and they were serially diluted in order to evaluate the viability of the bacteria liberated from the swollen tablets. The number of CFU was evaluated, as described above. The tests were performed in triplicate and the colonies were counted after aerobic incubation at 37° C. for at least 48 h.

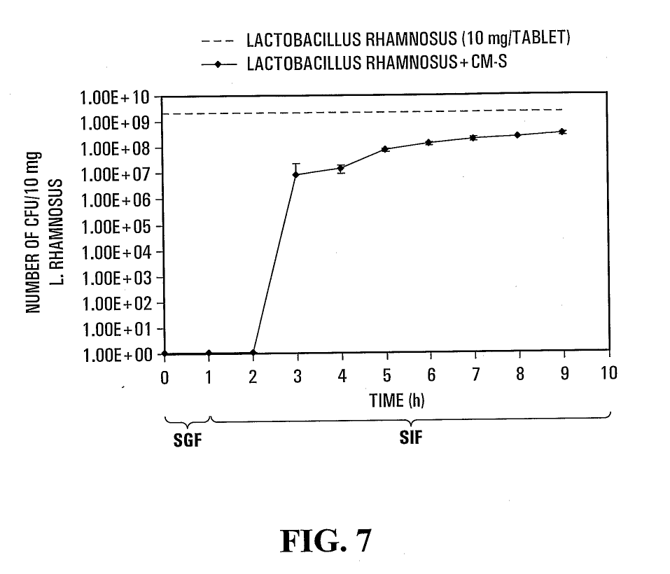

The release of the bacteria was clearly related to tablet swelling and dependent on the substitution degree. No viable bacteria were liberated after 1 h in SGF nor in the first hour in SIF (FIG. 7). The gel forming around the tablet, which may provide a mechanism for delayed liberation, could explain this lack of bacterial release. The gel would prevent access of water and α-amylase into the deeper layers of the tablet. A bacterial liberation from CM-S tablets was observed after 2 h in SIF. This liberation seems related to the swelling and erosion of the polymeric matrix. An increasing degree of substitution resulted in a decrease liberation of viable bacteria.

The exemplary data presented on a gram positive Lactobacillus rhamnosus are in good agreement with those obtained with the gram negative E. coli, showing the ability of this CM-S matrix to protect various bacterial species.

### Example 6

### Synthesis of Succinyl Starch (S-Starch)

A quantity of 70 g of Hylon VII (High Amylose Starch produced by National Starch, USA) was dissolved in 171 mL of distilled water. The solution was then heated at 50° C. under constant stirring for the remaining of the experiment. A solution of NaOH (13.7 g dissolved in 235 ml H₂O) was added to the starch solution. After 70 minutes, 130 ml of distilled water was added to the mixture and the pH was brought to 8.0 with acetic acid. The mixture was cooled down and the volume adjusted to 1.5 L with distilled water. Different variants of S-Starch were synthesized by slowly adding various amounts of solid succinic anhydride to the starch reaction medium while the pH is kept between 8.0 and 8.4. After the pH stabilisation the mixture was stirred for another 10 minutes.

A volume of 1.5 L of acetone was then gradually added to the mixture continuing the stirring for 20 minutes and the mixture was filtered on a filter paper. The resulting cake was crushed, transferred in an acetone/distilled water solution (3/2 v/v) and left under stirring for another 20 minutes. This process was repeated three times. Finally, the filtered powder was added to a solution containing only acetone and stirred for 20 minutes before being filtered again. The obtained powder was left to dry for 12 hours before being passed on a sieve to retain grains smaller than 300 μm.

Practically, five different S-starch products were obtained using 2, 4, 8, and 16 g of succinic anhydride to treat 70 g of Starch, in the mentioned conditions. The obtained S-Starch materials exhibited substitution degrees closely related to the ratio Succinic anhydride/Starch (exhibiting respectively 0.17, 0.30, 0.48 and 1 mEquiv. succinyl functions/g of S-Starch).

The S-starch used for Examples 7 and 8 of protein formulation presented a capacity of 1 mEquiv/g polymer.

### Example 7

### Determination of the Stability of α-Amylase Formulated with CM-Starch or S-Starch in Simulated Gastric Fluid

Tablets (200 mg), based on either CM-S or S-Starch, containing 10 mg of lyophilised α-amylase from Bacillus species (2560 units/mg protein) were formulated by direct compression of mixed powders at 3.0 T/cm². The tablets were then placed individually in 50 mL of simulated gastric fluid (SGF) pH 1.2 containing pepsin, for different times (0-120 min) at 37° C. (simulating the gastric passage), under agitation at 50 rpm, using an incubator shaker. The enzymatic activity was evaluated after 30, 60, 90 and 120 min. After the appropriate period of incubation in SGF, the tablets were transferred into 50 mL of Na₂HPO₄—NaH₂PO₄ buffer (pH 7.2, 50 mM) and crushed. This medium was diluted (1/50 v/v) and 1 ml of this diluted medium was used for determination of the enzymatic activity. As control for the alpha-amylase (active agent), 10 mg of free (non-formulated) enzyme was used in the same conditions.

The amylolytic activity was performed in triplicate and determined by the reductimetric method of Noelting and Bernfeld [1948] with dinitrosalicylic acid (DNS). Practically, 1 mL of the 1/50 dilution stated above was incubated for 3 min at room temperature with 1 mL of 1% soluble starch solution (pH 7.2, 10 mM) as substrate. Then, 1 mL of 1% DNS reagent (also stopping the enzymatic reaction) was added and the mixture was heated at once in a boiling water bath for 5 min to allow the released reducing sugars to react with DNS. The samples were then placed onto an ice water bath to stop the colorimetric reaction and the determination medium was diluted with 15 mL of distilled water, before reading absorbency at 535 nm.

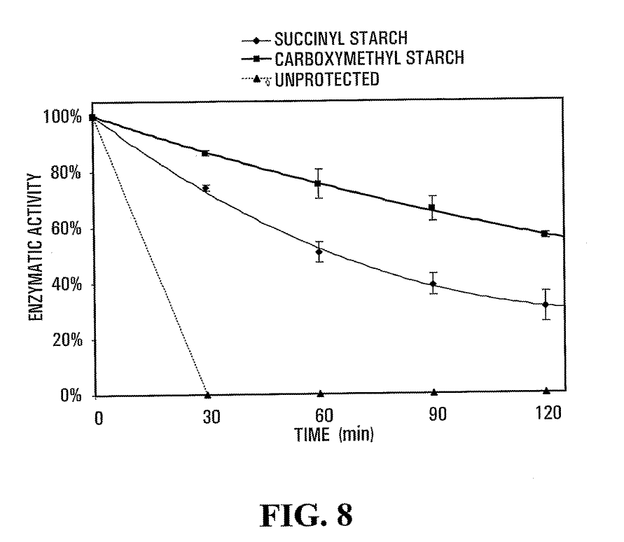

The enzymatic assays showed that, when formulated with CM-Starch or with S-Starch, both polymeric excipients were able to afford a considerable protection to the alpha-amylase enzyme against acidic denaturation for 120 min, whereas the free enzyme was totally inactivated (FIG. 8). After 120 min, the enzyme formulated with CM-Starch and S-Starch conserved 56% (CM-Starch) and 30% (S-Starch) of their initial activity, whereas for the free, unprotected α-amylase, no activity at all was observed after 30 min in SGF (pH 1.2). When formulated with CM-Starch, alpha-amylase activity presented a moderate decrease with time spent in SGF. In the case of S-Starch the decrease was more pronounced. However, it is worth to mention that, in both formulations, a considerable percentage of the enzyme activity was preserved, opening interesting perspectives for therapeutic formulations.

In conclusion, the CM- and S-Starch were found to afford a good enzyme protection, over a 120 min. gastric incubation.

Determination of Loading Capacity of CM-Starch or S-Starch with α-Amylase Active Agent

Tablets (200 mg), based on either CM-S or S-Starch, containing 10, 40, 80, 120 and 160 mg of α-amylase from Bacillus species (2560 units/mg protein) were formulated by direct compression at 3.0 T/cm². The tablets were then placed individually in 50 mL of simulated gastric fluid (SGF) pH 1.2 containing pepsin, for one hour at 37° C. under agitation at 50 rpm, using an incubator shaker. After the appropriate incubation period in SGF, the tablets were transferred into 50 mL of Na₂HPO₄—NaH₂PO₄ buffer (pH 7.2, 50 mM) and crushed. This medium was diluted (1/50 v/v) and 1 ml of this diluted medium was used for determination of the enzymatic activity. The amylolytic activity was determined by the method of Noelting and Bernfeld [1948] with dinitrosalicylic acid (DNS) as described in the previous example.

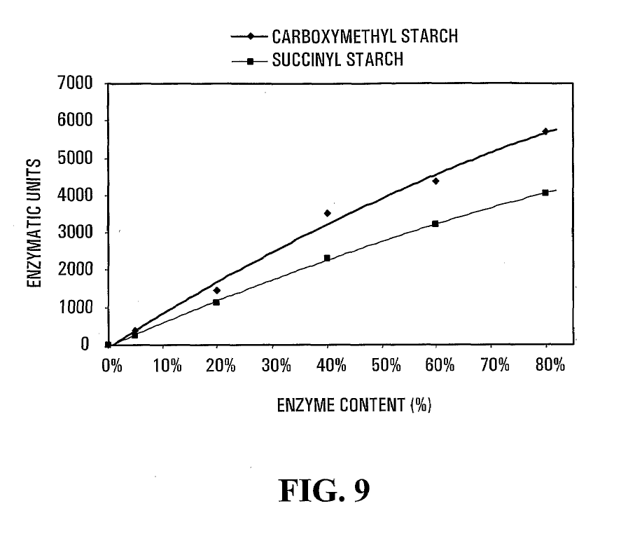

It was found, with both excipients, that tablets were in good shape and the alpha-amylase activity preserved after 1 h incubation in SGF, even when loaded with 80% alpha-amylase active agent (FIG. 9). These results demonstrate a high loading capacity of these pharmaceutical formulations, which is a desirable quality of an excipient: to ensure a high loading for low size of the solid dosage. A slightly higher stability was found for the CM-Starch formulations.

Determination of Liberation of α-Amylase Formulated with CM-Starch or S-Starch in pH 7.2 Solution.

Tablets (200 mg), based on either CM-Starch or S-Starch, containing 10 mg of α-amylase from Bacillus species (2560 units/mg protein) were formulated by direct compression at 3.0 T/cm². The tablets were then placed individually in 50 mL of simulated gastric fluid (SGF) pH 1.2 containing pepsin, for one hour at 37° C., under agitation (50 rpm), using an incubator shaker. After the appropriate the incubation period in SGF, the tablets were transferred into 50 mL of Na₂HPO₄—NaH₂PO₄ buffer (pH 7.2, 50 mM) and left into the incubator under the same conditions above. A volume of 50 μl was taken at different time to determine the enzymatic activity by the reductimetric method of Noelting and Bernfeld [1948] with dinitrosalicylic acid (DNS), as described in the previous examples.

The release of the alpha-amylase was related to tablet swelling and on the derivative type. Practically no enzymatic activities were found after 1 h of liberation in SGF nor in the first 30 min in phosphate buffer. The gel forming around the tablets, which may provide a mechanism for delayed liberation, could explain this lack of alpha-amylase release. The gel would prevent access of water into the deeper layers of the tablet. An alpha-amylase liberation from S-Starch tablets was observed after 1 h in phosphate buffer whereas, for CM-S, it was observed after 2 h (FIG. 10). This liberation seems related not only to the swelling but also to the erosion of the polymeric matrices.

### Example 8

### Determination of the Stability of Trypsin Formulated with CM-Starch or S-Starch in Simulated Gastric Fluid

Tablets (200 mg), based on either CM-S or S-Starch, containing 10 mg of trypsin from porcine pancreas (15500 units/mg protein) were formulated by direct compression at 3.0 T/cm². The tablets were then placed individually in 50 mL of simulated gastric fluid (SGF) pH 1.2 containing pepsin, for different times (0-120 min) at 37° C. (simulating the gastric passage), under agitation at 50 rpm, using an incubator shaker. The enzymatic activity was evaluated after 30, 60, 90 and 120 min in SGF. After the appropriate period of incubation, the tablets were-transferred into 50 mL of Na₂HPO₄—NaH₂PO₄ buffer (pH 7.2, 50 mM) and crushed. A volume of 20 μL of the solution was used for the measuring the enzymatic activity.

The trypsin activity was determined by the method of Bergmeyer, Gawehn and Grassl [1974] with Nα-Benzoyl-L-Arginine Ethyl Ester solution (BAEE). A volume of 20 μL of the solution above was added to 180 μL of water and incubated for 3 min at room temperature in 3 mL of BAEE solution (pH 7.6, 67 mM). The increase in A₂₅₃ₙₘ was recorded for 5 minutes and the ΔA₂₅₃ₙₘ/minute was used to determine the enzymatic activity.

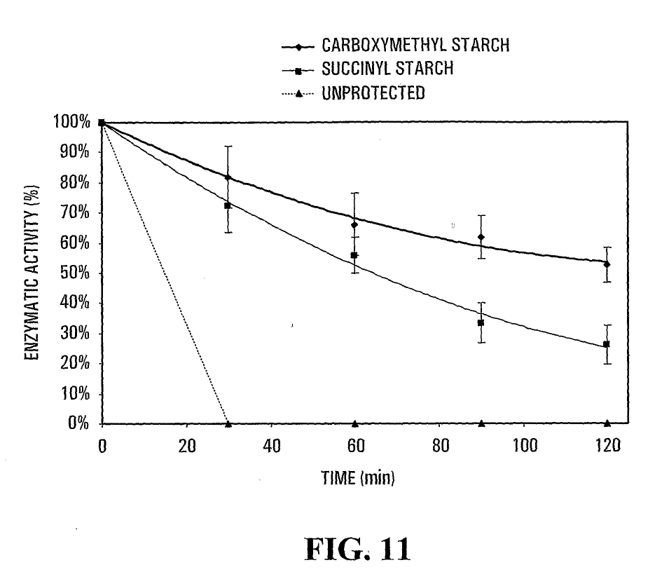

The enzymatic assays showed that, when formulated with CM-Starch or with S-Starch, both polymeric excipients were able to afford a considerable protection to the trypsin enzyme against acidic denaturation for 120 min, whereas the free enzyme was totally inactivated (FIG. 11). After 120 min, the enzyme formulated with CM-Starch and S-Starch conserved 52% (CM-Starch) and 26% (S-Starch) of their initial activity, whereas for the free, unprotected trypsin, no activity at all was observed after 30 min in SGF (pH 1.2). When formulated with CM-Starch, trypsin activity presented a moderate decrease with time spent in SGF. In the case of S-Starch the decrease was more pronounced. However, it is worth to mention that, in both formulations, a considerable percentage of the enzyme activity was preserved.

In conclusion, the CM- and S-Starch were found to afford a good enzyme protection, over 120 min gastric incubation.

Determination of Liberation of Trypsin Formulated with CM-Starch or S-Starch in pH 7.2 Solution.

Tablets (200 mg), based on either CM-S or S-Starch, containing 10 mg of trypsin from porcine pancreas (15500 units/mg protein) were formulated by direct compression at 3.0 T/cm². The tablets were then placed individually in 50 mL of simulated gastric fluid (SGF) pH 1.2 containing pepsin, for one hour at 37° C. under agitation at 50 rpm, using an incubator shaker. After the appropriate the incubation period in SGF, the tablets were transferred into 50 mL of Na₂HPO₄ NaH₂PO₄ buffer (pH 7.2, 50 mM) and left into the incubator under the same conditions above. A volume of 20 μl was taken at different times to determine the enzymatic activity.

The trypsin activity was determined by the method of Bergmeyer, Gawehn and Grassl [1974] with Nα-Benzoyl-L-Arginine Ethyl Ester solution (BAEE) as shown in the previous examples.

The release of the trypsin was related to tablet swelling and on the derivative type. Practically no enzymatic activity was detected after 1 h of incubation in SGF. In the case of CM-Starch, a low trypsin activity was detected even after 6 h of release in phosphate buffer (pH 7.2, 50 mM) whereas with the S-Starch excipient trypsin activity was detected from the moment of the tablet transfer and the release was completed within 2 h; subsequently, the activity gradually decreased, probably due to auto-proteolysis (FIG. 12).

Throughout this application, various references are referred to describe more fully the state of the art to which this invention pertains. The disclosures of these references are hereby incorporated by reference into the present disclosure.

### REFERENCES

Bergmeyer, H. U., Gawehn, K., and Grassl, M. (1974) Methods of Enzymatic Analysis (Bergmeyer, H. U., ed) Volume I, 2^{nd} ed., 515-516, Academic Press, Inc., New York, N.Y.
R. Edelman, R. G. Russell, G. Losonsky, B. D. Tall, C. O. Tacket, M. M. Levine, D. H. Lewis, Immunization of rabbits with enterotoxigenic E. coli colonization factor antigen (CFA/I) encapsulated in biodegradable microspheres of poly (lactide-co-lycolide). Vaccine, 11 (1993) 155-158.
J. C. deMan, M. Rogosa, and M. E. Sharpe, A medium for the cultivation of lactobacilli, J. Bacteriol. 23 (1960) 130.
Y. Dumoulin, S. Alex, P. Szabo, L. Cartilier, M. A. Mateescu, Cross-linked amylose as matrix for drug controlled release. X-ray and FT-IR structural analysis, Carbohydr. Polym. 37 (1998) 361-370.
P. Ispas-Szabo, F. Ravenelle, I. Hassan, M. Preda, M. A. Mateescu, Structure-properties relationship in cross-linked high-amylose stach for use in controlled drug release, Carbohydr. Res. 323 (2000) 163-175.
J. Kost, S. Shefer, Chemically-modified polysaccharides for enzymatically-controlled oral drug delivery, Biomaterials 11 (1990) 695-698.
J. Mulhbacher, P. Ispas-Szabo, V. Lenaerts, M. A. Mateescu—Cross-Linked high amylose starch derivatives as matrices for controlled release of high drug loadings. J. Control. Rel. 76 (2001) 51-58.
G. Noelting, P. Bernfeld, Diastatic enzymes. III. β-amylase. Determination of activity and control of absence of α-amylase, Helv. Chim. Acta 31 (1948) 286-290.
H. Roper, Applications of starch and its derivatives, Carbohydr. Eur. 15 (1996) 14-21.
H. D. Schell, M. Serban, M. A. Mateescu, T. Bentia, Acid and basic amylose ionic exchangers, Rev. Roumaine Chim. 23 (1978) 1143-1147.
G. W. Tannock, K. Munro, H. J. Harmsen, G. W. Welling, J. Smart, P. K. Gopal, Analysis of the fecal microflora of human subjects consuming a probiotic product containing Lactobacillus rhamnosus DR20. Appl Environ Microbiol 66 (2000) 2578-88.
U.S. Pharmacopeia National Formulary U.S.P. XXII, NF XVII, United States Pharmacopeial Convention Inc., Rockville, Md., 1990.
U.S. Pharmacopeia National Formulary U.S.P. XXIV, NF XVIX, United States Pharmacopeial Convention Inc., Rockville, Md., 2000.

1. A composition comprising:
(a) an uncrosslinked starch modified by an acidic group; and
(b) an agent;
wherein said composition is resistant to or substantially resistant to degradation in a first environment and is capable of degradation in a second environment, wherein the pKa of the acidic group is higher than the pH of the first environment and less than or equal to the pH of the second environment.

2. The composition according to claim 1, wherein the pH of the first environment is less than or equal to about 5.0.

3. The composition according to claim 2, wherein the pH of the first environment is from about 1.0 to about 5.0.

4. The composition according to claim 3, wherein the pH of the first environment is from about 1.2 to about 4.5.

5. The composition according to claim 1, wherein the first environment is located in the upper gastrointestinal tract of an animal.

6. The composition according to claim 5, wherein the first environment is within the stomach of the animal.

7. The composition according to claim 6, wherein the animal is a mammal.

8. The composition according to claim 7, wherein the mammal is a human.

9. The composition according to claim 1, wherein the pH of the second environment is greater than about pH 5.0.

10. The composition according to claim 9, wherein the pH of the second environment is greater than about pH 5.5.

11. The composition according to claim 10, wherein the pH of the second environment is from about 5.5 to about 8.0.

12. The composition according to claim 9, wherein the pH of the second environment is greater than about pH 5.8.

13. The composition according to claim 12, wherein the pH of the second environment is from about 5.8 to about 8.0.

14. The composition according to claim 9, wherein the pH of the second environment is greater than about pH 6.0.

15. The composition according to claim 14, wherein the pH of the second environment is from about 6.4 to about 7.2.

16. The composition according to claim 1, wherein the second environment is located in the lower gastrointestinal tract of an animal.

17. The composition according to claim 16, wherein the animal is a mammal.

18. The composition according to claim 17, wherein the mammal is a human.

19. The composition according to claim 16, wherein the second environment is the small intestine of the animal.

20. The composition according to claim 1, wherein the starch is high amylose starch.

21. The composition according to claim 20, wherein the starch contains more than about 70% amylose.

22. The composition according to claim 1, wherein the starch contains less than or equal to about 70% amylose.

23. The composition according to claim 22, wherein the starch contains from about 30% to about 70% amylose.

24. The composition according to claim 1, wherein the starch is selected from the group consisting of corn, wheat, bean, pea, rice, potato, cereal, root and tuber starch.

25. The composition according to claim 1, wherein the acidic group is selected from the group consisting of carboxyl, sulphate and phosphate groups.

26. The composition according to claim 25, wherein the carboxyl group is a succinyl group or a carboxyalkyl group.

27. The composition according to claim 26, wherein the alkyl is a lower alkyl.

28. The composition according to claim 27, wherein the lower alkyl is a C₁-C₆ alkyl.

29. The composition according to claim 28, wherein the C₁-C₆ alkyl is methyl and the acidic group is a carboxymethyl group.

30. The composition according to claim 1, wherein the degree of substitution of the starch with said acidic group is greater than or equal to about 0.1 mmol/g.

31. The composition according to claim 30, wherein the degree of substitution of the uncrosslinked starch with said acidic group is from about 0.1 mmol/g to about 4.0 mmol/g.

32. The composition according to claim 31, wherein the degree of substitution of the uncrosslinked starch with said acidic group is from about 0.1 mmol/g to about 1.25 mmol/g.

33. The composition according to claim 31, wherein the degree of substitution of the uncrosslinked starch with said acidic group is from about 0.6 mmol/g to about 4.0 mmol/g.

34. The composition according to claim 33, wherein the degree of substitution of the uncrosslinked starch with said acidic group is from about 0.6 mmol/g to about 1.25 mmol/g.

35. The composition according to claim 34, wherein the degree of substitution of the uncrosslinked starch with said acidic group is from about 0.6 mmol/g to about 0.8 mmol/g.

36. The composition according to claim 1, wherein the agent is selected from the group consisting of a drug, a polypeptide, an enzyme, an organelle, a microorganism, and a probiotic.

37. The composition according to claim 36, wherein the drug is a small molecule.

38. The composition according to claim 36, wherein the enzyme is a therapeutic enzyme.

39. The composition according to claim 38, wherein the therapeutic enzyme is a digestive enzyme.

40. The composition according to claim 39, wherein the digestive enzyme is a pancreatic enzyme.

41. The composition according to claim 40, wherein the pancreatic enzyme is α-amylase or trypsin.

42. The composition according to claim 36, wherein the microorganism is a prokaryote.

43. The composition according to claim 42, wherein the prokaryote is a bacterium.

44. The composition according to claim 43, wherein the bacterium is gram negative.

45. The composition according to claim 44, wherein the bacterium is Escherichia coli.

46. The composition according to claim 43, wherein the bacterium is gram positive.

47. The composition according to claim 46, wherein the bacterium is Lactobacillus sp.

48. The composition according to claim 47, wherein the bacterium is Lactobacillus rhamnosus.

49. The composition according to claim 1, wherein the composition is formulated in an oral dosage form or unit.

50. The composition according to claim 49, wherein the oral dosage form or unit is selected from the group consisting of a capsule, tablet, bead and a microsphere.

51. The composition according to claim 1, wherein the agent is in admixture with the uncrosslinked starch.

52. The composition according to claim 51, wherein the agent is substantially uniformly distributed throughout the composition.

53. The composition according to claim 1, wherein the composition comprises:
(a) a core portion comprising the agent; and
(b) a coat portion substantially covering the core portion, wherein the coat portion comprises the uncrosslinked starch.

54. The composition according to claim 53, wherein the core portion further comprises a pharmaceutically acceptable excipient.

55. The composition of claim 53, wherein the core portion further comprises an uncrosslinked starch modified with an acidic group.

56. The composition according to claim 55, wherein the degree of substitution of the uncrosslinked starch present in the coat portion is higher than the degree of substitution of the uncrosslinked starch present in the core portion.

57. A commercial package comprising:
(a) an uncrosslinked starch modified by an acidic group; and
(b) instructions for preparing the composition of claim 1.

58. The commercial package of claim 57, wherein said instructions set forth a method comprising:
(a) providing an agent; and
(b) combining the agent with the uncrosslinked starch thereby to obtain the composition.

59. A method of preparing a composition for selective release of an agent in a target environment, said method comprising:
(a) providing an uncrosslinked starch modified with an acidic group;
(b) providing an agent; and
(c) combining the uncrosslinked starch with the agent;
wherein the composition is resistant to or substantially resistant to degradation in a non-target environment and is capable of degradation in the target environment; wherein the pKa of the acidic group is higher than the pH of the non-target environment and less than or equal to the pH of the target environment.

60. The method of claim 59, wherein step (a) comprises modifying an uncrosslinked starch with an acidic group.

61. The method according to claim 60, wherein the modification step comprises reacting the uncrosslinked starch with a haloalkyl-substituted carboxylic acid.

62. The method according to claim 61, wherein the haloalkylsubstituted carboxylic acid is selected from the group consisting of monochloroacetic acid, 1-chloropropionic acid, 2-chloropropionic acid, chlorobutyric acid and succinic anhydride.

63. The method according to claim 60, wherein the modification step comprises reacting uncrosslinked starch with an anhydride.

64. The method according to claim 63, wherein the anhydride is succinic anhydride.

65. A composition prepared according to the method of claim 59.

66. A method for the selective delivery of an agent to a target environment comprising introducing the composition of claim 1 into the target environment.

67. A method for the selective delivery of an agent to a target environment comprising:
(a) providing the composition of claim 1 comprising the agent; and
(b) introducing the composition into the target environment.

68. The method of claim 67, wherein said providing step (a) comprises preparing a composition according to the method of claim 59.

69. The method of claim 67, wherein the target environment is the lower gastrointestinal tract of an animal.

70. The method of claim 69, wherein the environment is the small intestine of the animal.

71. The method according to claim 69, wherein the agent is administered orally.

72. The method of claim 69, wherein the animal is a mammal.

73. The method of claim 72, wherein the mammal is human.

74. The method according to claim 67, wherein the agent is selected from the group consisting of a drug, a” polypeptide, an enzyme, an organelle, a microorganism, and a probiotic.

75. A commercial package comprising:
(a) the composition of claim 1; and
(b) instructions for administering the composition to an animal.

76. The commercial package of claim 75, wherein the instructions are for delivering the agent to the lower gastrointestinal tract of an animal.

77. The commercial package of claim 76, wherein the lower gastrointestinal tract is the small intestine of the animal.

78. The commercial package of claim 75, wherein the instructions are for oral administration of the composition to an animal.

79. The commercial package of claim 75, wherein the animal is a mammal.

80. The commercial package of claim 79, wherein the mammal is human.

81-87. (canceled)

88. A composition comprising:
(a) an uncrosslinked starch modified by an acidic group; and
(b) a microorganism;
wherein said composition is resistant to or substantially resistant to degradation in a first environment and is capable of degradation in a second environment, wherein the pKa of the acidic group is higher than the pH of the first environment and less than or equal to the pH of the second environment.

89. The composition according to claim 88, wherein the acidic group is selected from the group consisting of carboxyl, sulphate and phosphate groups.

90. The composition according to claim 88, wherein the degree of substitution of the uncrosslinked starch with said acidic group is from about 0.6 mmol/g to about 0.8 mmol/g.

91. The composition according to claim 90, wherein the degree of substitution of the uncrosslinked starch with said acidic group is about 0.68 mmol/g.

92. The composition according to claim 88, wherein the microorganism is a prokaryote

93. The composition according to claim 92, wherein the microorganism is a bacterium.

94. The composition according to claim 88, wherein the microorganism is lyophilized.

95. A method for preserving viability of a microorganism, the method comprising combining the microorganism with an uncrosslinked starch modified by an acidic group, thereby forming a composition that is resistant to or substantially resistant to degradation in a first environment and is capable of degradation in a second environment, wherein the pKa of the acidic group is higher than the pH of the first environment and less than or equal to the pH of the second environment.

96. The method according to claim 95, wherein the acidic group is selected from the group consisting of carboxyl, sulphate and phosphate groups.

97. The method according to claim 95, wherein the degree of substitution of the uncrosslinked starch with said acidic group is from about 0.6 mmol/g to about 0.8 mmol/g.

98. The method according to claim 95, wherein the microorganism is a prokaryote.

99. The method according to claim 98, wherein the prokaryote is a bacterium.

100. The method according to claim 95, wherein the microorganism is lyophilized.

101. A commercial package comprising:
(a) an uncrosslinked starch modified by an acidic group, said uncrosslinked starch being capable of forming a composition that is resistant to or substantially resistant to degradation in a first environment and is capable of degradation in a second environment, wherein the pKa of the acidic group is higher than the pH of the first environment and less than or equal to the pH of the second environment; and
(b) instructions for preserving viability of a microorganism using said uncrosslinked starch.

102. The commercial package of claim 101, wherein said instructions set forth a method comprising:
(a) providing the microorganism; and
(b) combining the microorganism with the uncrosslinked starch thereby to obtain the composition.

103. The commercial package according to claim 101, wherein the microorganism is a lyophilized microorganism.

104. The commercial package according to claim 101, wherein the microorganism is a prokaryote.

105. The commercial package according to claim 104, wherein the prokaryote is a bacterium.

106. (canceled)
